(19) United States
(12) Patent Application Publication
(10) Pub. No.: US 2011/0305706 A1
(43) Pub. Date: Dec. 15, 2011

(54) Compositions and Methods for Treating a Disease Mediated by Soluble Oligomeric Amyloid Beta

(76) Inventors: Scott Thomas Brady, Chicago, IL (US); Gerardo Andres Morfini, Chicago, IL (US); Gustavo Pigino, Chicago, IL (US)

(21) Appl. No.: 13/202,838

(22) PCT Filed: Feb. 22, 2010
(86) PCT No.: PCT/US10/24893

§ 371 Date: Aug. 23, 2011

Related U.S. Application Data
(60) Provisional application No. 61/154,493, filed on Feb. 23, 2009.

(51) Int. Cl. A61K 39/395; A61P 25/28; C12N 5/02

(52) U.S. Cl. 424/146.1; 435/375

(57) ABSTRACT

The present invention relates to methods for restoring fast axonal transport in a cell affected by oligomeric amyloid beta and for treating an oligomeric amyloid beta-mediated disease such as Alzheimer's disease using a Casein Kinase 2 inhibitor and, in some embodiments, a Glycogen Synthase Kinase 3 inhibitor.

This application claims benefit of priority to U.S. Provisional Patent Application Ser. No. 61/154,493, filed Feb. 23, 2009, the content of which is incorporated herein by reference in its entirety.

### INTRODUCTION

This invention was made in the course of research sponsored by the National Institute of Neurological Disorders and Stroke (Grant Nos. NS23320, NS41170 and NS43408). The U.S. government has certain rights in this invention.

### BACKGROUND OF THE INVENTION

The complex functional changes and histopathology of Alzheimer disease (AD) make it one of the most challenging disorders faced by modern medicine. Histopathological hallmarks of AD include distinctive extracellular and intracellular aggregates of amyloid beta (Aβ) and tau (Maeda, et al. (2006) Neurosci. Res. 54(3):197-201; Terry & Davies (1980) Annu. Rev. Neurosci. 3:77-95). Synaptic dysfunction and axonopathy appear to be the earliest lesions in AD as corroborated by reduced immunoreactivity of synaptophysin and other synaptic markers in terminal fields of brain-affected areas (Masliah, et al. (1989) Neurosci. Lett. 103(2):234-239). AD affected neurons appear to die eventually as a consequence of synaptic dysfunction and loss, following a typical dying-back pattern of neuronal degeneration.

The amyloid hypothesis (Hardy & Higgins (1992) Science 256(5054):184-185), a dominant concept in AD research for many years, has been revised in recent years to include smaller soluble Aβ aggregates as playing an early and significant role in AD. Soluble oligomers of Aβ (oAβ) have been shown to be neurotoxic both in vivo and in vitro (Lambert, et al. (1998) Proc. Natl. Acad. Sci. USA 95(11):6448-6453; Dahlgren, et al. (2002) J. Biol. Chem. 277(35):32046-32053; Deshpande, et al. (2006) J. Neurosci. 26(22):6011-6018) as well as altering synaptic structure and function (Lacor, et al. (2007) J. Neurosci. 27(4):796-807). Moreover, oAβ levels correlate with impairments in cognitive function, learning and memory (Lesne, et al. (2006) Nature 440(7082):352-357; Cleary, et al. (2005) Nat. Neurosci. 8(1):79-84).

Intracellular Aβ was first described by Wertkin and colleagues (Wertkin, et al. (1993) Proc. Natl. Acad. Sci. USA 90(20):9513-9517). Immunogold electron microscopy showed that intraneuronal Aβ is pre- and post-synaptically enriched in both AD and Down syndrome's human brain and AD transgenic animal models, in association with dystrophic neurites and abnormal synaptic morphology (Gouras, et al. (2000) Am. J. Pathol. 156(1):15-20; Takahashi, et al. (2004) J. Neurosci. 24(14):3592-3599; Takahashi, et al. (2002) Am. J. Pathol. 161(5):1869-1879). Spatial and temporal analyses of intraneuronal oAβ accumulation show that it precedes plaque formation in both AD animal models and Down syndrome patients, suggesting that oAβ is an early intracellular toxic agent in AD (Takahashi, et/al. (2002) supra; Gyure, et al. (2001) Arch. Pathol. Lab. Med. 125(4):489-492). Aβ-induced neurodegeneration was seen in areas affected in AD such as cerebral cortex, hippocampus and amygdala, but absent in hindbrain and cerebellum of transgenic animals expressing intraneuronal Aβ (LaFerla, et al. (1995) Nat. Genet. 9(1):21-30). Similarly, transgenic flies expressing human wild-type (WT) or Arctic mutant E22G Aβ42 show neurodegeneration proportional to the degree of intraneuronal oAβ accumulation (Crowther, et al. (2005) Neuroscience 132(1):123-135). In addition, microinjection of heterogeneous Aβ42 into cultured human primary neurons at 1 pM concentration induced neuronal cell death (Zhang, et al. (2002) J. Cell Biol. 156(3):519-529).

Although Aβ is generated and accumulated in tissues other than brain (Joachim, et al. (1989) Nature 341(6239):226-230) neurons are selectively affected by intracellular Aβ (Zhang, et al. (2002) supra). This suggests that intracellular Aβ must disrupt a process essential for proper function and survival of neurons. Genetic, biochemical, pharmacological and cell biological research has shown that a reduction in fast axonal transport (FAT) is sufficient to trigger an adult-onset distal axonpathy and neurodegeneration. For example, point mutations affecting functional domains in kinesin or dynein motors can produce late-onset dying-back neuropathies in sensory or motor neurons (Hafezparast, et al. (2003) Science 300(5620):808-812; Reid, et al. (2002) Am. J. Hum. Genet. 71(5):1189-1194). Furthermore, dysregulation of FAT has been proposed as a pathological mechanism in several neurological disorders including AD (Morfini, et al. (2002) EMBO J. 23:281-293; Pigino, et al. (2003) J. Neurosci. 23:4499-4508), Kennedy's disease (Morfini, et al. (2006) Nat. Neurosci. 9:907-916; Szebenyi, et al. (2003) Neuron 40:41-52), Huntington's disease (Szebenyi, et al. (2003) supra) and Parkinson's disease (Morfini, et al. (2007) Proc. Natl. Acad. Sci. USA 104(7):2442-2447).

### SUMMARY OF THE INVENTION

The present invention features a method for restoring fast axonal transport in a cell affected by oligomeric amyloid beta by contacting the cell with an effective amount of an agent that inhibits Casein Kinase 2 activity. In some embodiments, the method further includes contacting the cell with a Glycogen Synthase Kinase 3 inhibitor.

The present invention also embraces a method for treating an oligomeric amyloid beta-mediated disease by administering to a subject in need of treatment an effective amount of a Casein Kinase 2 inhibitor. In accordance with one embodiment, the method further includes administering to the subject a Glycogen Synthase Kinase 3 inhibitor. In accordance with another embodiment, the oligomeric amyloid beta-mediated disease is Alzheimer's disease.

A kit for the treatment of an oligomeric amyloid beta-mediated disease is also provided, wherein said kit includes at least one Casein Kinase 2 inhibitor and at least one Glycogen Synthase Kinase 3 inhibitor.

### BRIEF DESCRIPTION OF THE DRAWINGS

FIG. 1 shows inhibition of FAT in both anterograde and retrograde directions by soluble oAβ. FAT rates were evaluated on isolated extruded axoplasms perfused with X/2 buffer containing synthetic heterogeneous 1-42 Aβ peptide (Aβ42, 2 μM). Perfusion with Aβ42 produced a significant reduction of FAT rates in both direction (FIG. 1A, n=3). Lines represent the best fit exponential of rates for vesicles moving in the anterograde (triangle) and retrograde (circle) direction of FAT. Each symbol represents a measurement of the rate for vesicles in the specified direction at a given time in an axoplasm. n represents the number of independent axons tested. To determine the effects of specific conformers of Aβ42, defined structural assemblies were employed. While neither unaggregated uAβ42 nor fibrillar fAβ had an effect on FAT at 100 nM, oligomeric oAβ42 (FIG. 1B, n=5) dramatically inhibited both directions of FAT at 100 nM. These results indicate that intracellular oAβ is a potent FAT inhibitor.

FIG. 2 shows that activation of endogenous casein kinase 2 (CK2) mediates oAβ42-induced FAT inhibition. Co-perfusion of oAβ (100 nM) with specific CK2 inhibitors such as DMAT (5 μM, n=4; FIG. 1A) or TBCA (200 nM, n=3; FIG. 1B) prevents FAT inhibition. Furthermore, co-perfusion of oAβ (100 nM) with a specific CK2 substrate peptide (500 μM, n=3; FIG. 1C) also prevents oAβ42-induced FAT inhibition. All these results indicate that oAβ42-induced FAT inhibition is mediated by activation of endogenous CK2 activity.

FIG. 3 shows that CK2 inhibits FAT and phosphorylates both KHC and KLC. CK2 (4 U, n=4) mimics the extent and inhibitory profile of oAβ42 on bidirectional FAT.

### DETAILED DESCRIPTION OF THE INVENTION

Analysis of the intraneuronal effects of different Aβ42 structural/conformation peptide assemblies on FAT in isolated squid axoplasms indicates that intracellular oAβ, but not intracellular uAβ (unaggregated amyloid beta) or fAβ (fibrillar amyloid beta), inhibits both anterograde and retrograde FAT at nanomolar concentrations. FAT inhibition results from activation of endogenous Casein Kinase 2 (CK2) by oAβ. The effect of oAβ on FAT can be prevented by two unrelated CK2 pharmacological inhibitors, 2-Dimethylamino-4,5,6,7-tetrabromo-1H-benzimidazole (DMAT) and tetrabromocinnamic acid (TBCA), as well as by an excess of a specific CK2 substrate peptide. Consistent with these data, perfusion of axoplasms with active CK2 induces a comparable inhibition of FAT. Both oAβ and CK2 increase KLCs phosphorylation by CK2, leading to kinesin-1 release from vesicular cargoes and inhibition of FAT. Therefore, modulation of CK2 activity finds application in restoring fast axonal transport and in the prevention and treatment of oAβ-mediated diseases. In so far as CK2 is a priming kinase for Glycogen Synthase Kinase 3 (GSK3) modification of KLC, particular embodiments of this invention embrace the use of a combination of one or more CK2 inhibitors with one or more GSK3 inhibitors.

Accordingly, the present invention is a method for restoring fast axonal transport in a cell affected by oAβ by inhibiting CK2 activity, and in some embodiments further inhibiting GSK3. For the purposes of the present invention, fast axonal transport is defined as kinesin- and dynein-mediated movement of mitochondria, lipids, synaptic vesicles, proteins, and other membrane-bound organelles and cellular components to and from a neuron's cell body through the axonal cytoplasm (the axoplasm) (Morfini, et al. (2006) In: Basic Neurochemistry (Ed. Siegel, et al.) pp. 485-502). Axonal transport is also responsible for moving molecules destined for degradation from the axon to lysosomes to be broken down. Axonal transport can be divided into anterograde and retrograde categories. Anterograde transport carries products like membrane-bound organelles, cytoskeletal elements and soluble substances away from the cell body toward the synapse and other axonal subdomains (Morfini, et al. (2006) In: Basic Neurochemistry (Siegel et al., ed.) Boston, Mass.: Elsevier Academic Press, pp. 485-502; Hirokawa & Takemura (2005) Nat. Rev. Neurosci. 6:201-14). Retrograde transport sends chemical messages and endocytosis products headed to endolysosomes from the axon back to the cell. In accordance with the disclosure provided herein, agents that inhibit CK2 activity, alone or in combination with a GSK3 inhibitor, prevent the inhibition of both anterograde and retrograde FAT in cells affected by intracellular oAβ.

Cells affected by intracellular oAβ include cells, in particular neurons, from a subject with an amyloid disease as well as neurons from a model system (e.g., an animal model or neuronal cell line) of an amyloid disease. In this regard, by exposing cells to the pathological amyloid protein, the cells undergo pathogenesis. Exposure of cells to oAβ can be by recombinant expression of exogenous Aβ by the cells, endogenous expression of Aβ, or injection or contact of cells with oAβ. Such methods of exposing cells to Aβ are routinely practiced in the art and any suitable method can be employed. In some embodiments, cells of the present invention are isolated (e.g., grown in vitro). In other embodiments, cells of the instant method are in vivo.

As is conventional in the art, amyloid beta protein (Aβ) is derived by the proteolytic processing of amyloid precursor protein (APP), resulting in a peptide predominantly 40 or 42 amino acids in length. Once produced, Aβ can aggregate and form soluble, oligomeric Aβ (oAβ), which is neurotoxic in vivo (Lambert, et al. (1998) Proc. Natl. Acad. Sci. USA 95:6448-6453; Hartley, et al. (1999) J. Neurosci. 19:8875-8884) and correlates with the degree of synaptic loss and cognitive impairment in Alzheimer's disease (Lue, et al. (1999) Am. J. Pathol. 155:853-862; McLean, et al. (1999) Ann. Neurol. 46:850-66). oAβ has been shown to alter LTP formation both in vitro and in vivo (Walsh, et al. (2002) Nature 416:535-539; Townsend, et al. (2006) J. Physiol. 572:477-92; Klyubin, et al. (2005) Nat. Med. 11:556-61) and decrease dendritic spine density and length in vitro (Calabrese, et al. (2007) Mol. Cell. Neurosci. 35:183-93; Lambert, et al. (1998) supra). For the purposes of the present invention, oAβ can be formed by oligomerization of Aβ39, Aβ40, Aβ41, Aβ42, or Aβ43, or combinations thereof, wherein these forms refer to Aβ peptide containing amino acid residues 1-39, 1-40, 1-41, 1-42, and 1-43. As a point of reference, the amino acid sequence of wild-type amyloid β 1-43 peptide is:

```
(SEQ ID NO: 1)
Asp-Ala-Glu-Phe-Arg-His-Asp-Ser-Gly-Tyr-Glu-Val-
  1 2 3 4 5 6 7 8 9 10 11 12
His-His-Gln-Lys-Leu-Val-Phe-Phe-Ala-Glu-Asp-Val-
  13 14 15 16 17 18 19 20 21 22 23 24
Gly-Ser-Asn-Lys-Gly-Ala-Ile-Ile-Gly-Leu-Met-Val-
  25 26 27 28 29 30 31 32 33 34 35 36
Gly-Gly-Val-Val-Ile-Ala-Thr
  37 38 39 40 41 42 43
```

In addition to wild-type forms of Aβ peptide, mutant Aβ peptide forms are also included within the scope of this invention. These include the Flemish (A21G) (Hendricks, et al. (1992) Nat. Genet. 1:218-221), Italian (E22K) (Tagliavini, et al. (1999) Alzheimer Rep. 2 (Suppl.) 28), Dutch (E22Q) (Levy, et al. (1990) Science 248:1124-1126; Van Broeckhoven, et al. (1990) Science 248:1120-1122), Arctic (E22G) (Nilsberth, et al. (2001) Nat. Neurosci. 4:887-893), and Iowa (D23N) (Grabowski, et al. (2001) Ann. Neurol. 49:697-705; Van Nostrand, et al. (2001) J. Biol. Chem. 276:32860-66) mutations, several of which have been shown to oligomerize in a manner similar to wild-type Aβ-(1-42) (Dahlgren, et al. (2002) J. Biol. Chem. 277:32046-53). Clinically, these mutations lead to AD-like symptoms secondary to intracerebral hemorrhage, although in some cases a progressive dementia is present in the absence of stroke.

In accordance with the present invention, fast axonal transport defects are corrected, restored or preserved in a cell by inhibiting or decreasing CK2 activity, and in some embodiments further inhibiting or decreasing GSK3 activity. In one embodiment of the present invention, inhibition of kinase activity is intended to mean that the inhibitor can decrease the activity of an isolated kinase enzyme (e.g., >90% pure) in an in vitro kinase assay. In another embodiment of the present invention, inhibition of kinase activity is intended to mean that the inhibitor specifically decreases expression of the kinase of interest thereby inhibiting the kinase activity. For example, RNAi or antisense molecules are routinely used in the art to specifically decrease expression of a protein of interest. Desirably, the kinase inhibitor selected has an IC₅₀ or Kᵢ of less to 10 μM, less than 1 μM less than 0.1 μM, or less than 0.01 μM.

An effective amount of an inhibitor of the invention is an amount that measurably decreases or inhibits any property (e.g., phosphorylation), biochemical activity (e.g., a kinase activity or an ability to bind to another protein) or biological activity possessed by the kinase of interest as compared to the kinase of interest when not contacted with the inhibitor. By inhibiting or decreasing kinase activity in accordance with the present invention, defects in fast axonal transport are restored or preserved.

As is known in the art, Casein Kinase 2 (CK2) is a serine/threonine protein kinase. The CK2 holoenzyme is a tetramer containing 2 alpha or alpha-prime subunits (or one of each) and 2 beta subunits, the sequences of which are conserved amongst mammalian species (Lozeman, et al. (1990) Biochemistry 29:8436-8447). The beta subunit is suggested to have a regulatory role in the holoenzyme, whereas the alpha subunit is the catalytic subunit. CK2 protein subunits are known in the art and described, e.g., under GENBANK Accession Nos. NP_{—}031814 (mouse alpha 1 polypeptide), NP_{—}034104 (mouse alpha prime polypeptide), NP_{—}001887 (human alpha prime polypeptide), NP_{—}034105 (mouse beta polypeptide), NP_{—}001311 (human beta polypeptide), NP_{—}001886 (human alpha 1 polypeptide).

Compounds for use in inhibiting or decreasing CK2 activity can be of any suitable type including chemical inhibitors, protein-based inhibitors, nucleic acid-based inhibitors, or mixtures thereof. The effectiveness of a CK2 inhibitor can be determined using the assay disclosed herein or any other known or commercially available assay, e.g., the CK2 assay kit marketed by Upstate Biotechnology.

Chemical inhibitors include small organic molecules such as tetrabromobenzimidazole/triazole derivatives and indoloquinazolines known to selectively inhibit CK2 activity. The structural basis for their selectivity is provided by a hydrophobic pocket adjacent to the ATP/GTP binding site, which in CK2 is smaller than in the majority of other protein kinases due to the presence of a number of residues whose bulky side chains are generally replaced by smaller ones. Examples of chemical CK2 inhibitors of use in the instant invention include, but are not limited to, 2-dimethylamino-4,5,6,7-tetrabromo-1H-benzimidazole (DMAT, Kᵢ=0.040 μM), tetrabromocinnamic acid (TBCA), 4,5,6,7-tetrabromo-1H-benzotriazole (TBB, Kᵢ=0.4 μM), 4,5,6,7-tetrabromo-1H-benzimidazole (TBBz), the emodin-related coumarinic compound 8-hydroxy-4-methyl-9-nitrobenzo[g]chromen-2-one (NBC, Kᵢ=0.22 μM), and the indoloquinazoline derivative [5-oxo-5,6-dihydroindolo-(1,2a)quinazolin-7-yl]acetic acid (IQA, Kᵢ=0.17 μM), CX-4945 (Cylene Pharmaceuticals), and K64 (3,4,5,6,7-pentabromo-1H-indazole) and K66 (1-carboxymethyl-2-dimethylamino-4,5,6,7-tetrabromo-benzimidazole) (Pagano, et al. (2008) Biochem. J. 415:353-365). Inorganic CK2 inhibitors have also been identified; these include the polyoxometalates (POMs) (Prudent et al. (2008) Chem. Biol. 15:683-692) that are aggregates of early transition metal ions and oxo ligands. POMs inhibit CK2 in the nanomolar range by targeting CK2α outside the ATP binding pocket.

Protein-based inhibitors include inhibitors that are composed of amino acid residues. Examples of protein-based inhibitors include peptide substrates, which mimic in vivo substrates thereby diluting CK2 activity, as well as antibodies that bind CK2 or CK2 substrates and block activation of CK2 or CK2 substrates. Exemplary protein-based inhibitors include the linear (IC₅₀=30 μM) and cyclic (IC₅₀=3.0 μM) Pc peptide (Gly-Cys-Arg-Leu-Tyr-Gly-Phe-Lys-Ile-His-Gly-Cys-Gly; SEQ ID NO:2) and its derivatives (Laudet, et al. (2007) Biochem. J. 408:363-73) as well as those disclosed in U.S. Pat. No. 7,374,767, incorporated herein by reference.

Nucleic acid-based inhibitors include molecules that inhibit the transcription or translation of CK2 mRNA. Examples of such inhibitors include RNAi or antisense molecules against the α or β subunits of CK2 such as those disclosed by Gu, et al. (2005) J. Biol. Chem. 280:27697-704 and Wang, et al. (2001) Mol. Cell. Biochem. 227:167-174, respectively.

In addition to CK2 inhibitors, certain embodiments of this invention embrace the additional inhibition of GSK3. GSK3 is known in the art as a multifunctional serine/threonine protein kinase implicated in the control of several regulatory proteins including glycogen synthase and transcription factors such as JUN. It also plays a role in the WNT and PI3K signaling pathways (see, e.g., Ali, et al. (2001) Chem. Rev. 101:2527-2540). In mammals, GSK3 is encoded by two genes, GSK3α and GSK3β, and the protein sequences encoded by these genes are described, e.g., under GENBANK Accession Nos. NP_{—}063937 (human GSK3α), NP_{—}001026837 (mouse GSK3α), NP_{—}059040 (rat GSK3α), NP_{—}002084 (human GSK3β), NP_{—}062801 (mouse GSK3β), and NP_{—}114469 (rat GSK3β).

GSK3 inhibitors for purposes of the present invention include chemical inhibitors, protein-based inhibitors, nucleic acid-based inhibitors and combinations thereof. The effectiveness of a GSK3 inhibitor can be determined using any known assay. See, e.g., Cross (2001) Meth. Mol. Biol. 124:147-159.

Chemical inhibitors of GSK3 include pyrroloazepines such as hymenialdisine (IC₅₀=0.010 μM); flavones such as flavopiridol (IC₅₀=0.450 μM), benzazepinones such as kenpaullone (IC₅₀=0.023 μM), alsterpaullone (IC₅₀=0.0.004 μM), and azakenpaullone (IC₅₀=0.018 μM), bis-indoles such as indirubin-3′-oxime (IC₅₀=0.022 μM), 6-bromoindirubin-3′-oxime (IC₅₀=0.005 μM), and 6-bromoindirubin-3′-acetoxime (IC₅₀=0.010 μM); pyrrolopyrazines such as aloisine A (IC₅₀=0.650 μM), and aloisine B (IC₅₀=0.750 μM); pyrazolopyridines such as pyrazolopyridine 18 (IC₅₀=0.018 μM) and pyrazolopyridine 34 (IC₅₀=0.007 μM); pyrazolopyridazines such as pyrazolopyridine 9 (IC₅₀=0.022 μM); aminopyrimidines such as CHIR98014 (IC₅₀=0.00065 μM for GSK3α and 0.00058 for GSK3β) and CHIR99021 (IC₅₀=0.010 μM for GSK3α and 0.007 for GSK3β); aminopyridines such as CT20026 (IC₅₀=0.004 μM); oxindoles such as SU9516 (IC₅₀=0.330 μM); thiazoles such as ARA014418 (IC₅₀=0.104 μM); bisindolylmaleimides such as staurosporine (IC₅₀=0.015 μM), compound 5a (IC₅₀=0.018 μM), GF109203x (IC₅₀=0.190 μM), and Ro318220 (IC₅₀=0.003-0.038 μM); azaindolylmaleimides such as compound 29 (IC₅₀=0.034 μM) and compound 46 (IC₅₀=0.036 μM); arylindolemaleimides such as SB216763 (IC₅₀=0.034 μM); anilinomaleimides such as SB415286 (IC₅₀=0.078 μM); and phenylaminopyrimidines such as CGP60474 (IC₅₀=0.010 μM). See, e.g., Meijer, et al. (2004) TRENDS Pharmacol. Sci. 25:471-480.

Protein-based inhibitors of GSK3 are well-known in the art and described, e.g., in WO 2009/016384.

Nucleic acid-based inhibitors of GSK3 include siRNA molecules, e.g., those available from Cell Signaling Technology, Origene, and Santa Cruz Biotechnology and described by Yu, et al. ((2003) Mol. Therapy. 7:228-36) as well as antisense molecules targeting GSK3 nucleic acids. Examples of suitable antisense molecules include those described by Ciaraldi, et al. ((2007) Endocrinology 148:4393-99) against GSK3α and those described by Takashima, et al. ((1995) Neurosci. Lett. 198:83-6) against GSK3β.

Additional kinase inhibitors can be identified and characterized using standard assays known in the art. For example, screening of chemical compounds for potent and selective inhibitors for CK2 has been carried by high-throughput docking (Vangrevelinghe, et al. (2003) J. Med. Chem. 46:2656-62). Furthermore a FRET-based microplate assay has been developed for human CK2 (Gratz, et al. (2009) doi: 10.3109/14756360903170038). Moreover, Vainshtein, et al. ((2002) J. Biomol. Screen. 7:507-14) describe a high-throughput, nonisotopic, competitive binding assay for protein kinase inhibitors.

Restoration of fast axonal transport in a cell finds application in research focusing on mechanisms of fast axonal transport and pathological amyloid protein activity as well as in the amelioration, delay, or prevention of oAβ-mediated diseases. Oligomeric Aβ-mediated diseases are a group of degenerative disorders characterized by cell/tissue damage caused by accumulation of soluble, toxic Aβ protein aggregates. Abnormal production, processing and/or clearance of Aβ leads to their accumulation and to the formation of cytotoxic, soluble oAβ. Diseases known to be mediated by oAβ include Alzheimer's disease (AD), which is characterized by a build-up of ADDLs, and AD-like diseases such as those mediated by mutant Aβ peptide forms described herein. See, also Ferreira, et al. (2007) IUBMB Life 59:332-45.

Treatment in accordance with the present invention includes administering, to a subject with an oAβ-mediated disease, an effective amount of a CK2 inhibitor, and optionally a GSK3 inhibitor, so that one or more signs or symptoms of the disease are ameliorated, delayed or prevented. In this regard, it is not necessary that the pathological oAβ be altered or degraded. However, in some embodiments, a CK2 inhibitor, and optionally a GSK3 inhibitor can be used in combination with an antibody or antibody fragment that binds specifically to oAβ. In particular embodiments, treatment results in the restoration of fast axonal transport and neuron function as compared to a subject not receiving such treatment.

Subjects benefiting from such treatment include humans as well as other animals that develop oAβ-mediated diseases. In this context, a subject is understood to include any mammalian species in which treatment of an oAβ-mediated disease is desirable, including agricultural and domestic mammalian species, as well as humans. The dosage ranges for the administration of the inhibitors of the invention can be in the range of about 0.1 mg/kg body weight to about 100 mg/kg body weight, or the limit of solubility of the active compound in a pharmaceutically acceptable carrier.

Kinase inhibitors, as described herein, can be used to prepare medicaments for treatment of an oAβ-mediated disease. The inhibitors can be included in pharmaceutical compositions useful for practicing the therapeutic method described herein. Pharmaceutical compositions of the present invention contain a physiologically acceptable carrier together with one or more kinase inhibitors as described herein, dissolved or dispersed therein as an active ingredient. In a particular embodiment, the pharmaceutical composition is not immunogenic when administered to a mammalian patient, such as a human, for therapeutic purposes.

Kinase inhibitors can be administered subcutaneously, intravenously, parenterally, intraperitoneally, intradermally, intramuscularly, topically, enteral (for example, orally), rectally, nasally, buccally, vaginally, by inhalation spray, by drug pump or via an implanted reservoir in dosage formulations containing conventional non-toxic, physiologically (or pharmaceutically) acceptable carriers or vehicles.

In a specific embodiment, it may be desirable to administer the inhibitor of the invention locally to a localized area in need of treatment; this can be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers. When it is desirable to direct the inhibitor to the central nervous system, techniques which can opportunistically open the blood brain barrier for a time adequate to deliver the drug there through can be used. For example, a composition of 5% mannitose and water can be used.

Suitable pharmaceutically acceptable carriers include but are not limited to water, salt solutions (for example, NaCl), alcohols, gum arabic, vegetable oils, benzyl alcohols, polyethylene glycols, glycerol, gelatin, carbohydrates such as lactose, amylose or starch, magnesium stearate, talc, silicic acid, viscous paraffin, perfume oil, fatty acid esters, hydroxymethylcellulose, polyvinyl pyrolidone, and combinations thereof. The pharmaceutical preparations can be sterilized and if desired, mixed with auxiliary agents, for example, lubricants, preservatives, stabilizers, wetting agents, emulsifiers, salts for influencing osmotic pressure, buffers, coloring, flavoring and/or aromatic substances and the like which do not deleteriously react with the active agents.

The compositions, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The compositions can be formulated in accordance with the routine procedure as a pharmaceutical composition adapted for intravenous administration to subjects. Typically, compositions for intravenous administration are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

For topical application, there are employed as nonsprayable forms, viscous to semi-solid or solid forms including a carrier compatible with topical application and having a dynamic viscosity preferably greater than water. Suitable formulations include, but are not limited to, solutions, suspensions, emulsions, creams, ointments, powders, enemas, lotions, sols, liniments, salves, aerosols, etc., which are, if desired, sterilized or mixed with auxiliary agents, for example, preservatives, stabilizers, wetting agents, buffers or salts for influencing osmotic pressure, etc. The drug may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

When used in combination, the CK2 inhibitor(s) and GSK3 inhibitor(s) can be provided in the form of a kit. In one embodiment, the kit includes one or more vials respectively containing one or more CK2 inhibitors and one or more vials containing one or more GSK3 inhibitors. In another embodiment, the CK2 inhibitor(s) and GSK3 inhibitor(s) are premixed in a single vial. The kit can further include a means for administration, such as a syringe or pump, and instructions for the administration of the inhibitors. Alternatively, the CK2 inhibitor(s) and GSK3 inhibitor(s) are each in individual syringes or premixed in a single syringe with appropriate instructions provided for administration. In a further embodiment, the instructions will describe the methods disclosed herein.

The amount of inhibitor which will be effective in the treatment of a particular oAβ-mediated disease may be dependent upon the disease, age of the subject, health status of the subject and the like, and can be determined by standard clinical techniques. In addition, in vitro or in vivo assays can optionally be employed to help identify optimal dosage ranges. The precise dose to be employed in the formulation will also depend on the route of administration, and the seriousness of the disease or disorder, and should be decided according to the judgment of the practitioner and each subject's circumstances.

The invention is described in greater detail by the following non-limiting examples.

### Example 1

### Materials and Methods

Antibodies and Reagents. Antibodies used in this analysis included H2 monoclonal antibody (mAb) against kinesin-1 heavy chains (Deboer, et al. (2008) Biochemistry 47:4535-4543); 63-90 mAb that preferentially recognizes dephosphorylated kinesin-1 light chains (Morfini, et al. (2002) supra); and TrkB rabbit polyclonal antibody from Santa Cruz Biotechnology. Protein kinase inhibitors were from Calbiochem including SB 203580, CK2 inhibitors (DMAT and TBCA, tetrabromocinnamic acid). Inhibitors were diluted in DMSO or ethanol as appropriate and kept at −80° C. until used. CK2 substrate peptide was obtained from Sigma. Active CK2 and Antarctic Phosphatase were from New England Biolabs.

Preparation of Aβ42 Solutions. For heterogeneous Aβ solutions, synthetic Aβ42 peptide (Bachem) was prepared as described (Pigino, et al. (2001) J. Neurosci. 21(3):834-842). These heterogeneous Aβ solutions were perfused into extruded axoplasms isolated from the squid Loligo pealeii at a concentration of 2 μM. Vesicle motility was evaluated for 50 minutes after perfusion. For defined solutions of unaggregated, oligomeric and fibrillar Aβ42 conformations, synthetic Aβ42 from California Peptide, Inc. were meticulously prepared (Stine, et al. (2003) J. Biol. Chem. 278(13):11612-11622). Solutions of defined Aβ42 conformations were perfused as above at 100 nM.

Atomic Force Microscopy. Peptide solutions were characterized using a NanoScope IIIa scanning probe work station equipped with a MultiMode head using a vertical engage E-series piezoceramic scanner (Veeco, Santa Barbara, Calif.). AFM probes were single-crystal silicon microcantilevers with 300-kHz resonant frequency and 42 Newton/meter spring constant model OMCL-AC160TS-W2 (Olympus). Twenty μl of sample solution (diluted from 100 μM to 10 μM (or 30 μM for fibrils) in deionized MilliQ water) was spotted on freshly cleaved mica, incubated at room temperature for 3 minutes, rinsed with 0.02 μm of filtered (Whatman Anotop 10) MilliQ water (Millipore), and blown dry with tetrafluoroethane (CleanTex MicroDuster III). For Aβsolutions in F12 media, the cleaved mica was pre-treated with 3 μl of 1 M HCl and rinsed prior to addition of sample. Some samples were imaged under dry helium. Image data were acquired at scan rates between 1 and 2 Hz with drive amplitude and contact force kept to a minimum. Data were processed to remove vertical offset between scan lines by applying zero order flattening polynomials using Nanoscope software (Version 5.31r1, Veeco).

Motility Studies in Isolated Axoplasm. Axoplasms were extruded from giant axons of the squid L. pealeii provided by Marine Biological Laboratory according to known methods (Brady, et al. (1985) Cell Motility 5:81-101). Vesicle motility was analyzed using a ZEISS AXIOMAT microscope equipped with DIC optics. Vesicle velocities were assessed in accordance with conventional methods (Brady, et al. (1993) Meth. Cell Biol. 39:191-202).

Immunochemical Methods. Proteins were separated by SDS-PAGE on 4-12% Bis-Tris gels (NuPage minigels, Invitrogen), using MOPS Running Buffer (Invitrogen) and transferred to PVDF membranes (Pigino, et al. (2001) supra). Immunoblots were blocked with 5% non-fat dried milk, in phosphate-buffered saline, pH 7.4, and probed with different polyclonal and monoclonal antibodies. Primary antibody binding was detected with HRP-conjugated anti-mouse and anti-rabbit secondary antibody (Jackson Immunoresearch) and visualized by chemiluminescence (ECL, Amersham). Kinesin immunoprecipitation was performed from mouse brain according to published procedures (Morfini, et al. (2006) In: Methods in Molecular Biology, ed Sperry A (Humana Press, Clifton, N.J.), Vol 392, pp 51-70). Isolation of membrane vesicle fractions from axoplasms was as previously described (Lapointe, et al. (2009) J. Neurosci. Res. 87(2):440-451). Two axoplasms from the same animal were prepared and incubated with appropriate effectors (CK2 buffer, active CK2, uAβ or oAβ) and vesicle fractions evaluated by immunoblot using H2 and Trk antibodies. Trk served as protein loading control and vesicle fraction marker.

In vitro Kinesin-1 Phosphorylation. Immunoprecipitated kinesin-1 was either phosphorylated by CK2 (250 U) or was previously dephosphorylated by Antartic Phosphatase (15 U) following NEB protocols and then phosphorylated by CK2. Kinase reactions were initiated by addition of 100 μM of radiolabeled ATP. After 1 hour at 30° C., reactions were stopped by addition of 60 μl sample buffer. Proteins were separated by SDS-PAGE, gels dried and exposed to a phosphoroimager screen for analysis (Morfini, et al. (2006) supra).

Statistical Analysis. All experiments were repeated at least 3 times. Unless otherwise stated, the data was analyzed by two sample t-test for pairwise comparisons or ANOVA followed by post-hoc Student-Newman-Keul's test in order to make all possible comparisons. Data was expressed as mean±SEM and significance was assessed at p<0.05 or 0.01 as noted.

### Example 2

### oAβ is a Potent Inhibitor of FAT

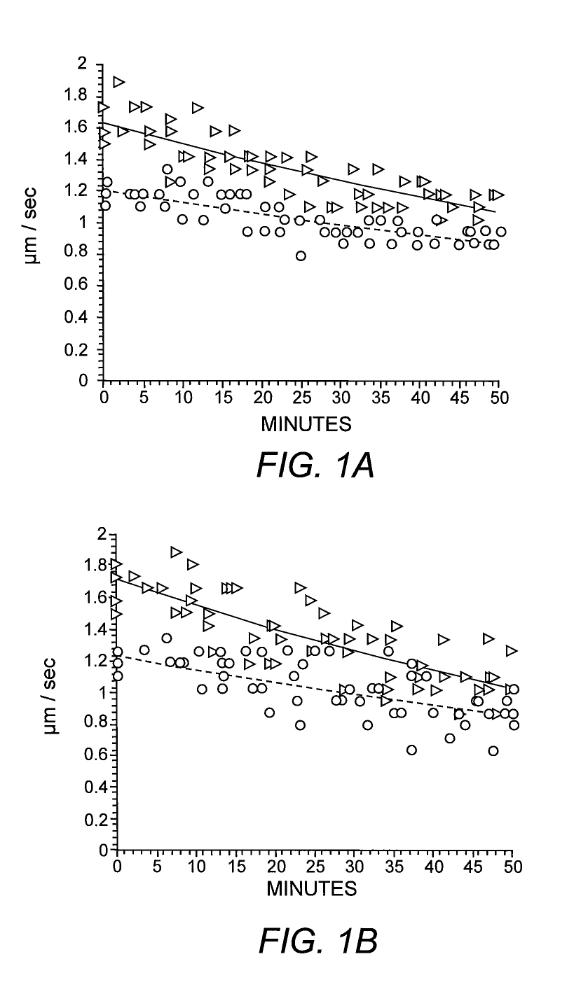

Previous studies have shown reduced anterograde FAT of specific synaptic cargoes in different AD mouse models known to accumulate intracellular Aβ in the axonal compartment progressively (Pigino, et al. (2003) supra). To evaluate the intraxonal effects of Aβ on FAT directly, heterogeneous preparations of synthetic Aβ42 were perfused into isolated extruded axoplasms dissected from the squid L. pealeii. Perfusion of synthetic Aβ42 at 2 μM severely reduced both kinesin-1-based anterograde and cytoplasmic dynein (cDyn)-based retrograde FAT (FIG. 1A). Reconstitution of synthetic Aβ in aqueous solutions produces a mixture of different Aβ assemblies due to preexisting molecular structures in lyophilized stocks (Stine, et al. (2003) supra). To determine the contribution of specific Aβ structural assemblies to inhibition of FAT, Aβ42 was prepared under defined conditions that yield uniform solutions of oligomers or fibrils (Stine, et al. (2003) supra). These unaggregated, oligomeric and fibrillar Aβ42 preparations (100 nM) were perfused into isolated axoplasms to evaluate the effects on FAT perfused into isolated extruded axoplasms at 100 nM. Neither uAβ nor fAβaltered FAT in either direction. In contrast, perfusion of 100 nM oAβ inhibited both directions of FAT significantly (FIG. 1B) and inhibition of FAT was seen as low as 10 nM oAβ. Collectively, these results indicate that oAβ has a toxic activity that inhibits FAT when present intracellularly. This toxic effect is independent of transcription and translation, since extruded axoplasms are separated from cell bodies. The oAβ inhibition of FAT is also independent of energy metabolism changes, because 5 mM ATP is added to assays of vesicle motility with oAβ, thereby ruling out the possibility that oAβ affects FAT by altering mitochondrial ATP production.

### Example 3

### oAβ-Induced Fat Inhibition Results from Endogenous CK2 Activation

To determine the specific molecular mechanism by which oAβ inhibits FAT, the characteristic profile of FAT inhibition was evaluated. The ability of nanomolar levels of oAβ to inhibit FAT indicated that the molecular mechanism of inhibition was likely to be enzymatic (Morfini, et al. (2006) supra) rather than a physical blockade. Abnormal phosphorylation of different neuronal cytoskeletal proteins is a characteristic hallmark in AD and previous studies in isolated axoplasm have identified multiple kinase and phosphatase activities that can inhibit FAT (Morfini, et al. (2002) supra; Morfini, et al. (2006) supra; Morfini, et al. (2007) supra; Morfini, et al. (2001) Dev. Neurosci. 23:364-376). A number of different phosphotransferase activities are altered in brains of AD patients, AD animal models and cell lines exposed to Aβ. The list of abnormal kinase activities in AD is extensive, among them several unrelated serine/threonine protein kinases including GSK3 (Pigino, et al. (2003) supra; Morfini, et al. (2002) Neuromol. Med. 2(2):89-99; Pei, et al. (1997) J. Neuropathol. Exp. Neurol. 56:70-78), p 38 (Pei, et al. (2001) J. Alzheimers Dis. 3(1):41-48), JNK (Pei, et al. (2001) supra), cdk5 (Lee & Tsai (2003) J. Alzheimers Dis. 5(2):127-137; Morfini, et al. (2004) EMBO J. 23:2235-2245), and casein kinase 2 (CK2) (Masliah, et al. (1992) Am. J. Pathol. 140(2):263-268), wherein many of these enzymes are involved in regulation of FAT (Morfini, et al. (2001) supra; Morfini, et al. (2002) supra).

Similarities in the profile of FAT inhibition produced by activation of several members of the stress-activated protein kinases (SAPK) and oAβ (Morfini, et al. (2006) supra) led to an analysis of the role of axonal SAPK in oAβ-induced FAT inhibition. Co-perfusion of homogeneous oAβ42 with 5 μM SB203580, a well-characterized inhibitor for multiple SAPKs (Morfini, et al. (2006) supra), did not prevent inhibition FAT by oAβ, making a role for endogenous SAPKs unlikely.

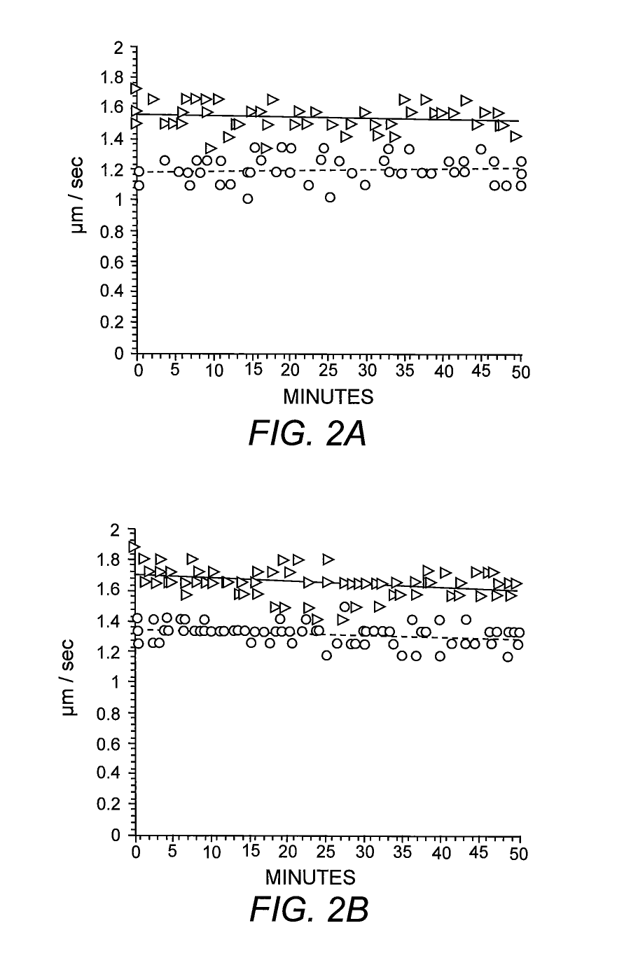

CK2 is another kinase that can inhibit both anterograde and retrograde FAT (Morfini, et al. (2001) supra). Based on results showing that heterogeneous Aβ could directly stimulate CK2 kinase activity in vitro (Chauhan, et al. (1993) Brain Res. 629(1):47-52), the role of endogenous CK2 activation in oAβ-induced FAT inhibition was evaluated. 2-Dimethylamino-4,5,6,7-tetrabromo-1H-benzimidazole (DMAT) is a potent and highly specific ATP-competitive inhibitor of CK2 (Pagano, et al. (2004) Biochem. Biophys. Res. Commun. 321(4):1040-1044) derived from 4,5,6,7-Tetrabromo-2-azabenzimidazole (TBB). Co-perfusion of oAβ42 with DMAT (FIG. 2A) blocked the oAβ inhibitory effects on both directions of FAT entirely. A newly developed CK2 inhibitor also derived from TBB, tetrabromocinnamic acid (TBCA), has proven to be an even more specific for CK2 (Pagano, et al. (2007) Chembiochem. 8(1):129-139) and TBCA also blocked inhibition of FAT by oAβ (FIG. 2B). Finally, a peptide substrate specific for CK2 was used as a competitive inhibitor of CK2 activity and it also prevented inhibition of FAT by oAβ (FIG. 2C). Thus, selective inhibition of CK2 can prevent oAβ-induced inhibition of FAT.

### Example 4

### Perfusion of oAβ Increases Phosphorylation of Squid Kinesin-1 Light Chains by Endogenous CK2 Activity

To assess the ability of CK2 to phosphorylate a molecular motor for FAT, the phosphorylation status of squid kinesin-1 was evaluated after oAβ perfusion using the 63-90 monoclonal antibody against kinesin-1 light chain (KLCs) (Stenoien & Brady (1997) Mol. Biol. Cell 8:675-689). The 63-90 antibody preferentially recognizes dephosphorylated KLCs (Morfini, et al. (2002) supra; Pigino, et al. (2003) supra; Pigino, et al. (2009) supra). KLCs in immunoprecipitates (IP) from mouse brain were recognized by 63-90 and immunoreactivity increased after treatment with Antarctic phosphatase. When kinesin-1 IP were treated with CK2, KLCs immunoreactivity was dramatically reduced, consistent with a CK2 phosphorylation site within the 63-90 epitope. When the extent of endogenous squid KLCs phosphorylation was evaluated by immunoblot with 63-90, KLC immunoreactivity was significantly reduced in axoplasms perfused with oAβ relative to uAβ perfused axoplasms. These data indicate that oAβ activates endogenous CK2 activity leading to a concomitant increase in squid KLC phosphorylation.

### Example 5

### CK2 Mimics the Inhibitory Effect of oAβ on FAT by Directly Phosphorylating Kinesin-1

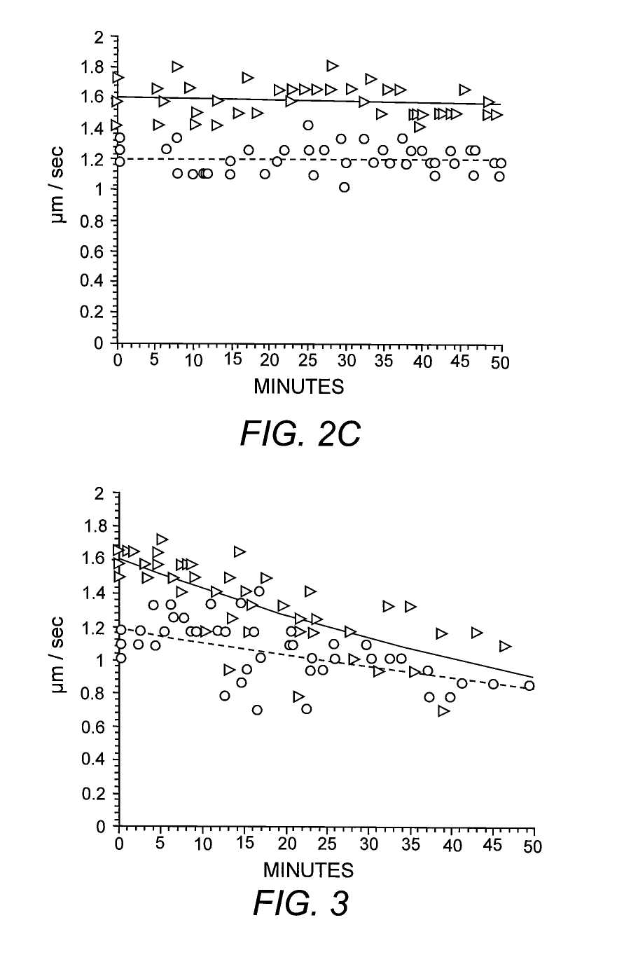

If oAβ effects on FAT were due to activation of CK2, then recombinant CK2 should have the same effect on FAT when perfused into axoplasm. Indeed, both directions of FAT were inhibited by CK2 and the inhibition seen with CK2 was qualitatively indistinguishable from oAβ effects on FAT (FIG. 3). In vitro kinase assays showed that both KHC (kinesin-1 heavy chain) and KLCs from mouse brain were phosphorylated by recombinant CK2, consistent with previous results (Donelan, et al. (2002) J. Biol. Chem. 277(27):24232-24242). Together these experiments indicate that inhibition of FAT by oAβ is mediated by activation of endogenous CK2 and phosphorylation of kinesin-1.

### Example 6

### oAβ-Induced FAT Inhibition Results from Kinesin-1 Release of its Vesicular Cargoes

Phosphorylation of kinesin-1 by various kinases can regulate its activities and affect anterograde FAT (Morfini, et al. (2002) supra; Morfini, et al. (2006) supra; Morfini, et al. (2001) supra). Previous studies on phosphorylation of KLC at the 63-90 epitope suggest that this site regulates binding of kinesin-1 to vesicle cargoes (Pigino, et al. (2003) supra; Lapointe, et al. (2009) supra). To determine whether oAβ leads to release of kinesin-1 from cargoes, vesicle fractions were isolated by flotation assays from axoplasms treated for 50 minutes with either uAβ or oAβ and the association of kinesin-1 with membrane fractions was evaluated by immunoblot with the kinesin-1 KHC-specific antibody H2. A dramatic reduction of kinesin-1 immunoreactivity was seen on axonal vesicles from oAβ—relative to uAβ-perfused axoplasms. There was a comparable reduction of kinesin-1 immunoreactivity on axonal vesicles perfused with active CK2 relative to those perfused with CK2 buffer control. An antibody to the integral membrane protein Trk (Moreno, et al. (1998) Proc. Natl. Acad. Sci. USA 95(25):14997-15002) was used to verify presence of vesicles and serve as a loading control. Collectively, these results indicate that one mechanism by which oAβ mediates FAT inhibition is by increasing CK2 activity and phosphorylating KLCs, leading to release of kinesin-1 from vesicles.

### Example 7

### Implications of oAβ-Induced FAT Inhibition in Alzheimer's and Alzheimer's-Like Diseases

A number of observations indicate that intracellular oAβ plays a role in Alzheimer's disease pathogenesis. For example, oAβ has been observed in association with membranous organelles as well as in the cytoplasm of neuronal processes either in association with MTs or in empty areas within axonal processes (Takahashi, et al. (2004) supra). Furthermore, oAβ is reported to be toxic when introduced into the cytosol of cultured primary neurons (Zhang, et al. (2002) supra). The results herein provide direct evidence that intraneuronal Aβ is a strong inhibitor of both anterograde and retrograde FAT. Based upon analysis of different conformational forms of Aβ, only oAβ inhibited FAT at concentrations that were physiologically plausible (10-100 nM). The ability of soluble oAβ to inhibit FAT at stoichiometries an order of magnitude or more lower than affected molecular motors effectively eliminated the possibility that oAβ represented steric interference.

Perfusion of isolated axoplasms with active GSK3 (Morfini, et al. (2002) supra), JNK (Morfini, et al. (2006) supra) or CK2 (Morfini, et al. (2001) supra) results in inhibition of FAT. Two different mechanisms have been identified for inhibition of kinesin-based FAT. When KLCs are phosphorylated by GSK3 (Morfini, et al. (2002) supra; Pigino, et al. (2003) supra; Lapointe, et al. (2009) supra), binding of kinesin-1 to cargoes is reduced. Alternatively, phosphorylation of KHC by JNK inhibits the ability of kinesin-1 to bind microtubules (Morfini, et al. (2006) supra). Neuropathogenic forms of Huntingtin (Htt) or androgen receptor (AR) increase activity of JNK and inhibition of bidirectional FAT in extruded axoplasms (Morfini, et al. (2006) supra; Szebenyi, et al. (2003) supra). This profile of FAT inhibition is similar to oAβeffects, but co-perfusion of oAβ with SB203580, an inhibitor of JNK and p38 kinases, does not block effects of oAβ on FAT.

While GSK3 inhibits anterograde FAT (Morfini, et al. (2002) supra), the results herein demonstrate that oAβaffects both anterograde and retrograde FAT. Previous studies in axoplasm have indicated that CK2 can also inhibit both directions (Morfini, et al. (2001) supra). Therefore, it was determined whether coperfusion of axoplasms with oAβ and selective CK2 inhibitors (DMAT or TBCA) could block the effects of oAβ on FAT. The results indicate that these inhibitors completely block the effects of oAβ on FAT. To eliminate the possibility that a secondary kinase target was responsible for oAβ effects, axoplasms were co-perfused with oAβ and a CK2-specific peptide substrate, which served as a competitive inhibitor of axoplasmic CK2 substrates and prevented inhibition FAT. These results indicate that oAβ-induced FAT inhibition results from activation of endogenous CK2.

CK2 phosphorylates both KHC and KLCs in vitro (Donelan, et al. (2002) supra), and phosphorylation of endogenous KLCs in axoplasm was shown herein to significantly increase by perfusion of either CK2 or oAβ as determined by reduced immunoreactivity with the phospho-sensitive 63-90 monoclonal antibody. Previous studies with GSK3 showed that the 63-90 epitope was also sensitive to GSK3 activity (Morfini, et al. (2002) supra; Pigino, et al. (2003) supra; Lapointe, et al. (2009) supra). Significantly, CK2 was a priming kinase for GSK3 modification of KLC and GSK3 did not phosphorylate KLC without a priming phosphorylation (Morfini, et al. (2002) supra), indicating that activation of CK2 in the presence of active GSK3 enhances the effects of GSK3 on kinesin-1 based motility.

The functional consequence of GSK3-mediated phosphorylation of KLC is release of kinesin-1 from its vesicle cargoes, (Morfini, et al. (2002) supra; Pigino, et al. (2003) supra; Lapointe, et al. (2009) supra). Consistent with this observation, the amount of kinesin-1 on axoplasmic vesicles was substantially reduced relative to controls with both oAβ and CK2 perfused axoplasms. Thus, phosphorylation of KLCs by CK2 activity leads to release of kinesin-1 from cargoes, either directly or in combination with endogenous GSK3 activity. These experiments indicate that intraneuronal oAβ leads to abnormal activation of axonal CK2, which phosphorylates kinesin-1 subunits, the KLCs, leading to removal the anterograde motor from vesicles and inhibition of FAT. However, unlike GSK3, which affects only anterograde FAT, CK2 and oAβ inhibit both directions of FAT, indicating that CK2 affects cDyn function as well.

Although hyperphosphorylation of neuronal proteins in AD has long been recognized, the question of how specific kinases are activated in AD has not been extensively addressed. The activation of CK2 by oAβ provides a partial answer to this critical question in AD pathogenesis. These data identify kinesin-1 and cDyn as key targets of CK2 in AD. By affecting the function of motors critical for FAT and maintenance of neuronal connectivity, a direct link from oAβ to neuronal degeneration is defined.

Effective therapeutic intervention in progressive neurological disorders depends on a clear understanding of the molecular mechanisms associated with that disease. It has now been shown that dysregulation of CK2 by oAβ is capable of inhibiting the vital neuronal process of FAT. Therefore, pharmacological regulation of CK2 activity represents a novel target for therapeutic intervention in AD, particularly when combined with treatments that manage GSK3 activity as well.

What is claimed is:

1. A method for restoring fast axonal transport in a cell affected by oligomeric amyloid beta comprising contacting the cell with an effective amount of an agent that inhibits Casein Kinase 2 activity thereby restoring fast axonal transport in the cell.

2. The method of claim 1, further comprising contacting the cell with a Glycogen Synthase Kinase 3 inhibitor.

3. A method for treating an oligomeric amyloid beta-mediated disease comprising administering to a subject in need of treatment an effective amount of Casein Kinase 2 inhibitor thereby treating the subject's oligomeric amyloid beta-mediated disease.

4. The method of claim 3, further comprising administering to the subject a Glycogen Synthase Kinase 3 inhibitor.

5. The method of claim 3, wherein the oligomeric amyloid beta-mediated disease is Alzheimer's disease.

6. A kit for the treatment of an oligomeric amyloid beta-mediated disease comprising one or more Casein Kinase inhibitor and one or more Glycogen Synthase Kinase 3 inhibitor.


SEQUENCE LISTING (2 sequences)

SEQ ID NO: 1 - protein, 43 aa, Homo sapiens
DAEFRHDSGYEVHHQKLVFFAEDVGSNKGAIIGLMVGGVVIAT

SEQ ID NO: 2 - protein, 13 aa, Artificial sequence
  Synthetic peptide
GCRLYGFKIHGCG